(19) United States
(12) Patent Application Publication
(10) Pub. No.: US 2021/0401557 A1
(43) Pub. Date: Dec. 30, 2021

(54) ORAL MEDICAL ASSIST DEVICE

(71) Applicant: Shu-Chuan SHEU, Ashburn, VA (US)

(72) Inventor: Shu-Chuan SHEU, Ashburn, VA (US)

(21) Appl. No.: 16/930,311

(22) Filed: Jul. 15, 2020

(30) Foreign Application Priority Data
Jun. 24, 2020 (TW) 109121627

(51) Int. Cl. A61C 17/06; A61C 5/82

(52) U.S. Cl. CPC A61C 17/06; A61C 5/82

(57) ABSTRACT

An oral medical assist device for covering an oral area of a patient, including a mask body, is provided. The mask body, is at least partially transparent and defines an internal space, including a suction connecting portion and an operation portion. The suction connecting portion and the operation portion are configured to opposite to each other. The operation portion includes at least one opening. The suction connecting portion is configured to connect to a medical suction device to absorb objects in the internal space while the mask body covers the patient's oral area. Another oral medical assist device includes a closed end, so that the droplets from the oral area will not be spread outside of the mask body. In this way, the oral medical assist device can reduce or prevent droplets from spreading to the environment when the patient opens his/her mouth.

### CROSS-REFERENCES TO RELATED APPLICATIONS

This application claims priority to Taiwan Patent Application No. 109121627 filed on Jun. 24, 2020, the disclosures of which are incorporated herein by reference in their entirety.

### BACKGROUND OF THE INVENTION

### Field of the Invention

The present invention relates to an oral medical assist device, and in particular, relates to an oral medical assist device which can cover an oral area of a patient to reduce or avoid the spread of droplets.

### Descriptions of the Related Art

When people talk and breathe, droplet particles will be generated. During oral-related examination or treatment in medical processes, patients need to open their mouths wide, and a lot of droplets will be generated at this time. Although medical personnel can wear mouth-muffles and masks to reduce the risk of bacterial and viral infection, the droplets will still splash and spread for several meters and stick to articles in the medical environment, which results in possible contact and air infection.

In particular, high-speed handpiece, ultrasonic scaling machine, air-water syringe and other instruments used in dental treatment will produce a large amount of aerosols during treatment, and make the liquid with saliva and toxins of removed old fillings splash into the air. If the patient contains pathogens that can be transmitted through droplets and contact, the infection risk of medical personnel and other personnel in the same space will be greatly increased, and the inhalation of toxins by medical personnel or patients will further affect health thereof.

However, at present, in the epidemic prevention measures of oral-related treatment, most medical personnel wear heavy protective clothing or multi-layer protective equipment (goggles, multi-layer mouth-muffles, etc.), and frequently clean various tools and environment, or even expensive negative-pressure clinics are set up. No corresponding equipment is available to reduce or stop droplets and aerosols overflowing from the mouth of patients at low cost during treatment, and it is impossible to further avoid contact or air infection and/or inhalation of toxins by medical personnel and patients due to the spread and attachment of the droplets to objects in the environment.

Even if there are attempts to redevelop saliva suction devices, such new devices are usually bulky and/or expensive, which makes it impossible to popularize them quickly to achieve the expected epidemic prevention effect. If the existing suction device is modified with available hardware parts, it will encounter many problems, such as failing to conform to the mouth shape, poor material and/or blocking and interfering with medical sight.

Accordingly, an urgent need exists in the art to improve the above drawbacks. It shall be additionally noted that, the above technical content is used to facilitate understanding of the problem to be solved by the present invention, which is not necessarily disclosed or well known in the art.

### SUMMARY OF THE INVENTION

An object of the present invention is to provide an oral medical assist device, which can reduce or prevent droplets from widely spreading in the air when a patient opens his/her mouth during treatment.

To achieve the aforesaid objective, an oral medical assist device provided by the present invention comprises a mask body which is at least partially transparent and defines an internal space. The mask body comprises a suction connecting portion and an operation portion, the suction connecting portion and the operation portion are configured to be opposite to each other, and the operation portion comprises at least one opening. The suction connecting portion is configured to connect to a medical suction device to absorb objects (including droplets, aerosols, debris and splashing liquid, etc.) near the internal space when the mask body covers the oral area of the patient.

To achieve the aforesaid objective, another oral medical assist device provided by the present invention comprises a mask body which is at least partially transparent and defines an internal space. The mask body comprises a closed end and an open end opposite to the closed end. An outer edge of the open end of the mask body abuts against a face of the patient when the mask body covers the oral area, so that droplets from the oral area will not be spread outside of the mask body.

In an embodiment, the mask body comprised in the oral medical assist device of the present invention is made of a transparent material and has a thickness that is not greater than 3.0 mm and not less than 0.1 mm.

In an embodiment, the suction connecting portion of the mask body comprised in the oral medical assist device of the present invention comprises a tube, and a diameter of the tube decreases gradually along an axial direction.

In an embodiment, at least one opening of the operation portion comprised in the oral medical assist device of the present invention has an outer edge, the outer edge is streamlined, and a portion of the outer edge extends toward the suction connecting portion.

In an embodiment, the mask body comprised in the oral medical assist device of the present invention has a shape of an oblique cone.

In an embodiment, the open end comprised in the oral medical assist device of the present invention may comprise at least two openings.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 to FIG. 2C are respectively a schematic view of use, a schematic perspective view, a schematic spread-out view and a schematic bottom cross-sectional view of an oral medical assist device according to a first preferred embodiment of the present invention;

FIG. 3A to FIG. 3B are respectively a partial and perspective schematic view and a schematic spread-out view of the oral medical assist device in different fixation modes according to the first preferred embodiment of the present invention;

FIG. 4 to FIG. 5 are respectively a schematic view of use and a schematic perspective view of an oral medical assist device according to a second preferred embodiment of the present invention;

FIG. 6A and FIG. 6B are partial and perspective schematic views of a suction connecting portion in different connection modes according to the second preferred embodiment of the present invention;

FIG. 7 is a schematic view of a suction connecting portion without a tube according to the second preferred embodiment of the present invention;

FIG. 8 to FIG. 9 are respectively a schematic perspective view and a schematic spread-out view of the opening located at different positions according to the second preferred embodiment of the present invention;

FIG. 10 is a schematic perspective view of the second preferred embodiment of the present invention without an opening;

FIG. 11 is a schematic perspective view according to a third preferred embodiment of the present invention; and

FIG. 12 is a schematic view of the limited splash range of an oral medical assist device according to a second preferred embodiment of the present invention.

### DESCRIPTION OF THE PREFERRED EMBODIMENT

Hereinafter, specific embodiments according to the present invention will be specifically described. However, the present invention may be practiced in many different forms of embodiments without departing from the spirit of the present invention, and the scope claimed in the present invention should not be interpreted as being limited to what stated in the specification.

Unless the context clearly indicates otherwise, singular forms “a” and “an” and the like as used herein also include plural forms. In addition, the orientations (such as front, back, left, right, upper, lower, inside and outside, etc.) are relative orientations, which may be defined according to the use state of the oral medical assist device, and do not indicate or imply that the oral medical assist device needs to be constructed or operated in a specific direction, and the orientations should not be understood as limitation to the present invention.

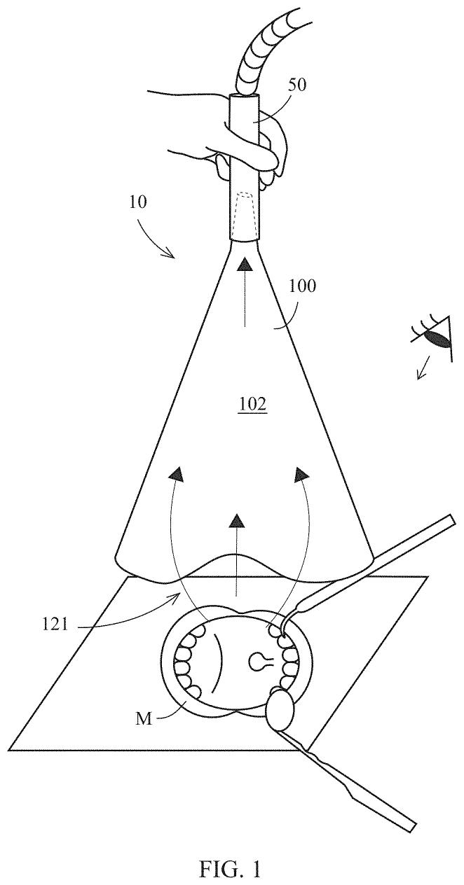

Please refer to FIG. 1, which is a schematic perspective view according to a first preferred embodiment of the present invention. An oral medical assist device 10 (which is called for short as an assist device 10 hereinafter) according to the first preferred embodiment of the present invention may also be called a droplet vacuumer or a droplet trapper, and comprises a mask body 100, and it may be used as a dental medical assist device. The oral medical assist device is hold by a medical personnel (a doctor or an assistant) with hands or fixed by a commercially available clamping bracket (not shown) and placed above an oral area M of a lying patient, and it is partially transparent for the medical personnel to watch the oral cavity of the patient through the assistive device 10. The detailed technical contents are described as follows.

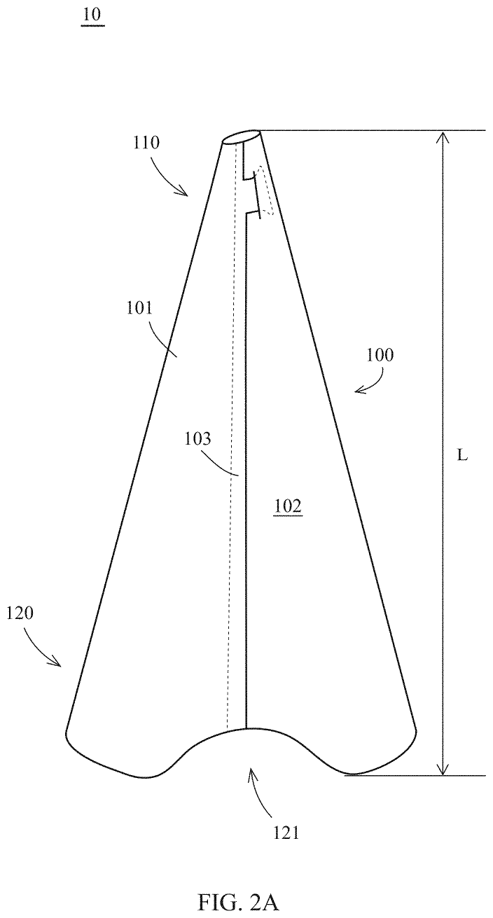

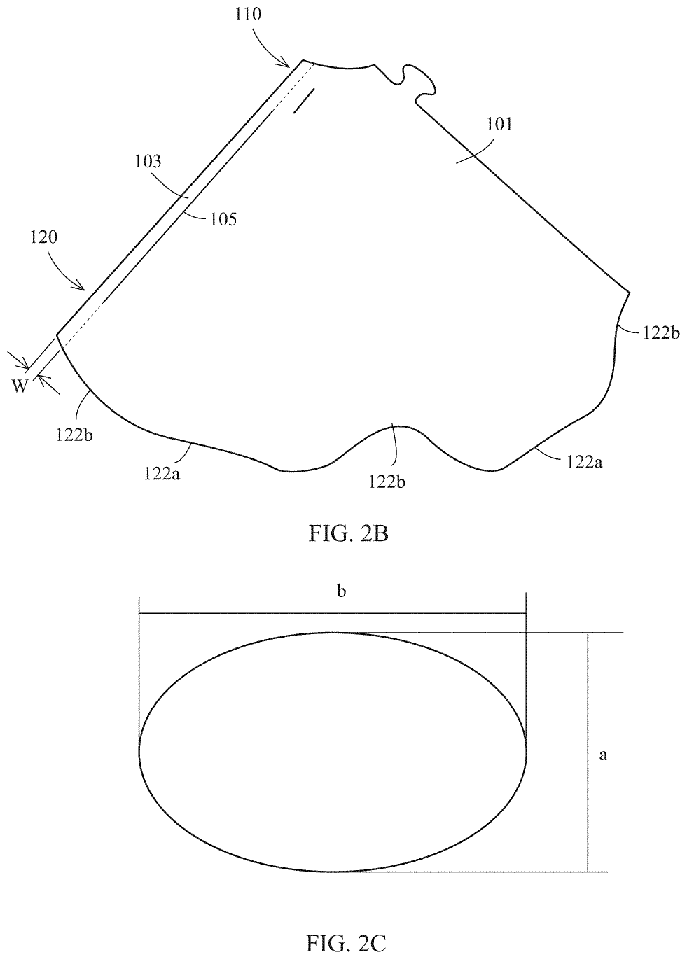

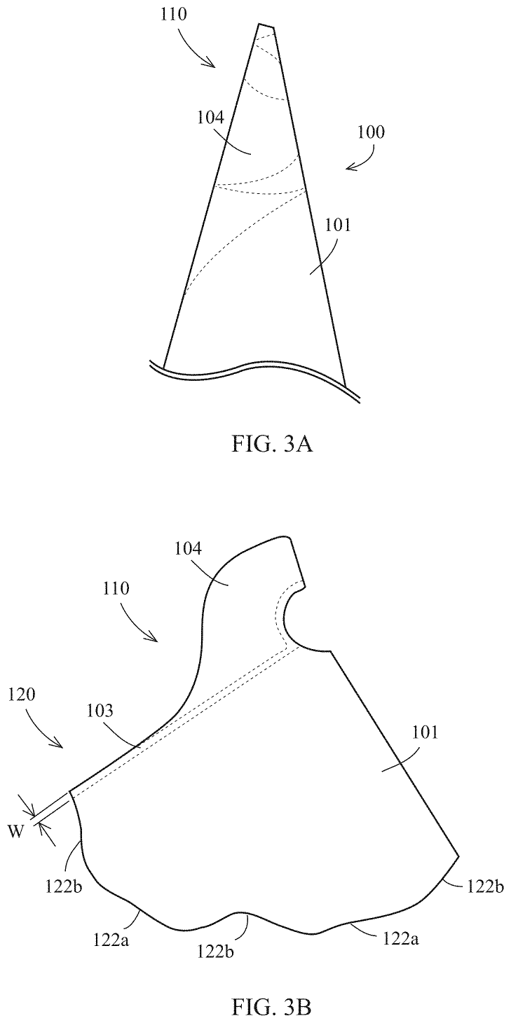

Referring to FIG. 2A to FIG. 3B and taking a mask body 100 which is transparent as a whole as an example, the mask body 100 may be composed of an annular outer wall 101, and it defines an internal space 102 and comprises a suction connecting portion 110 and an operation portion 120. The outer wall 101 may be made of polypropylene (PP), polycarbonate (PC), polylactic acid (PLA), polyethylene terephthalate (PET) or polyvinyl chloride (PVC) or the like, and may have a thickness approximately uniform. The thickness may be not greater than 3.0 mm so as not to affect the inner diameter of the opening of the suction connecting portion 110 (described later in detail). Further speaking, the thickness may be not greater than 0.3 mm and not less than 0.1 mm in order to reduce the aberration caused by light refraction when the medical personnel watches the oral cavity through the outer wall 101. In addition to the above materials, any materials that can make the finished product light and transparent can be used, and materials that are resistant to alcohol contact or can undergo other disinfection and sterilization processes are preferred.

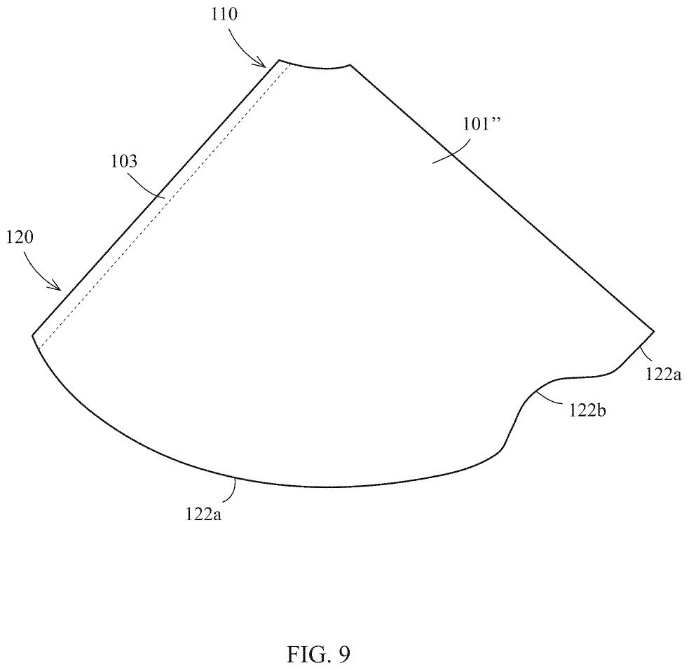

When the mask body 100 is transparent as a whole, it may be integrally manufactured by injection molding, or a sheet prototype (as shown in FIG. 2B, FIG. 3B, and FIG. 9) may be obtained by laser cutting, die cutting or the like, and then fixed into a cone shape by a fixing portion after being curled, and the fixing portion may be corresponding mortise and tenon, buttons, and/or an adhesive structure. For example, the mortise and tenon may be implemented as at least one lug along an axial direction of the cone-shaped mask body 100 and at least one corresponding cutting opening (a lug and a notch are shown in the upper end of FIG. 2). The button may be for example a plastic snap button, and an opposite slot on the planes. The adhesive structure may, for example, comprise an adhesive layer 103 (adhesive, adhesive strip, tape, etc.). The above means may be applied alone or in combination with each other. For example, as shown in FIG. 2A to FIG. 2B, when the mask body 100 is made of a sheet prototype, portions which are difficult to fix due to the resilience of materials or portions which do not affect the medical sight (the upper end of the mask body 100 shown in the figure) may selectively be combined with the mortise and tenon or the button and the adhesive structure, while other parts (the lower end of the mask body 100 shown in the figure) may adopt adhesive alone. In this way, the mask body 100 can be made more stable without affecting medical treatment. Alternatively, as shown in FIG. 3A to FIG. 3B, the fixing portion may further comprise a protruding portion 104, which may have an inverted-hook shape and be located in the internal space 102 after being curled, and the force-bearing range of the mask body 100 after being curled may be increased. In this way, the shape of the mask body 100 can be stabilized even when using the adhesive structure alone (not limited to the shape shown in the figure). When the adhesive structure is used, a groove 105 (as shown in FIG. 2B) may be provided at an edge of the predetermined lamination of the sheet prototype (i.e., an edge of the adhesive layer 103) to facilitate the alignment during fixing, and the groove 105 preferably does not extend to an opening of the suction connecting portion 110 and/or the operation portion 120 so as not to affect the shape of the opening.

When using the mask body 100, the fixing portion may be located on the left, right, opposite or proximal sides of the doctor (i.e., the left, right, rear sides or upper end of the mask body 100) so as not to affect the doctor's view of the patient's oral cavity through the front side, inclined front side or near the bottom end of the mask body 100. When the fixing portion of the mask body 100 comprises means that may block the medical sight (e.g., buttons, opacity adhesive, adhesive strip), a width W of the fixing portion may be not greater than 1 cm and not less than 0.5 cm (as measured along the tapered outer surface) so as not to affect the sight of medical personnel and provide sufficient stability. When the fixing portion of the mask body 100 does not comprise means that may block the medical sight (e.g., the sheet prototype is transparent as a whole, and it is fixed with transparent tape after being curled), the width of the fixing portion is not limited thereto. Without affecting medical treatment, the mask body 100 may also be only partially transparent (not shown), and may still provide the functions of watching and examining the oral cavity.

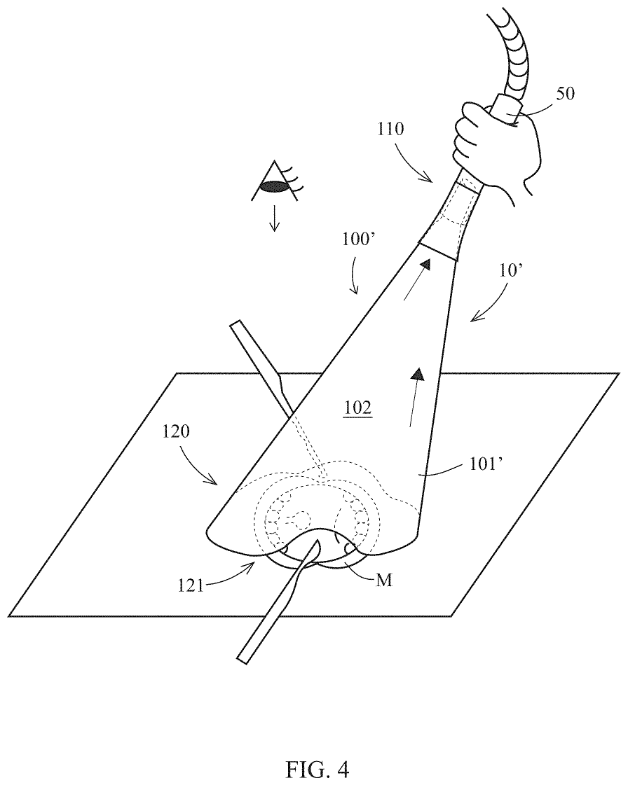

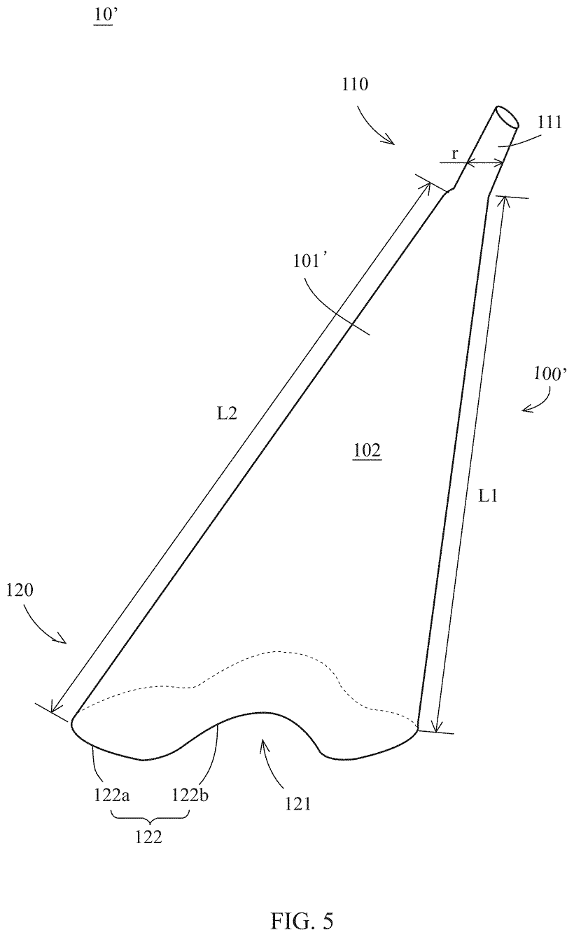

As shown in FIG. 4, which is an assist device 10′ according to a second preferred embodiment of the present invention, the mask body 100′ may also be composed of an annular outer wall 101′ similar to the outer wall 101, defines an internal space 102 and comprises a suction connecting portion 110 and an operation portion 120. The mask body 100 may further has a shape of an oblique cone, which inclines in a direction away from the doctor's view when it is in use, so that light or sight of the doctor is less likely to be blocked by the personnel or equipment fixing the suction connecting portion 110 when the doctor watches the oral cavity of the patient through the assist device 10. The conical bottom sections of the mask bodies 100, 100′ may all be elliptical, and according to the research and literature of human mouth size in various countries, the short diameter a thereof may be not less than 7 cm, and the long diameter b thereof may be not less than 15 cm (as shown in FIG. 2C). In this way, the mask body is suitable for the size of most people's mouths when opened, and completely covers the oral area of the patients (which may include the philtrum but exclude the nose) during medical treatment, and is also suitable for the suction capability of most medical suction devices. In addition, referring to FIG. 2A and FIG. 5, the mask body 100 may have a length L of not less than 17 cm or not greater than 25 cm. When the mask body 100′ has a shape of an oblique cone, the conical short side may have a length L1 which is not less than 17 cm or not greater than 25 cm, and the conical long side may have a length L2 which is preferably 2 cm longer than the length L1, thereby increasing the slope of the mask body 100, and preventing the personnel or equipment (fixing clips or pipelines, etc.) for fixing the suction connecting portion 110 from interfering with medical treatment. Moreover, moisture will not be accumulated on the surface of the internal space 102 too quickly for the mask body 100 and then block the sight of the doctor and generate water drops. With the above size limitation, the inner diameter of the opening at the top of the suction connecting portion 110 of the mask body 100′ made of sheet prototype will be affected by the thickness of the outer wall 101′, so the thickness is preferably not greater than 3.0 mm.

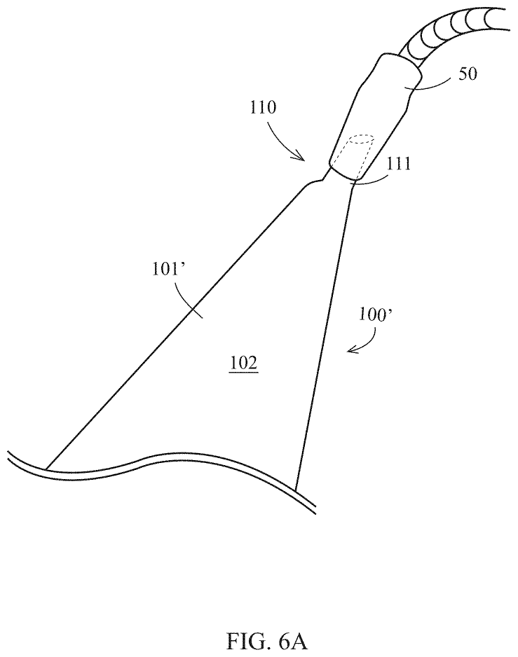

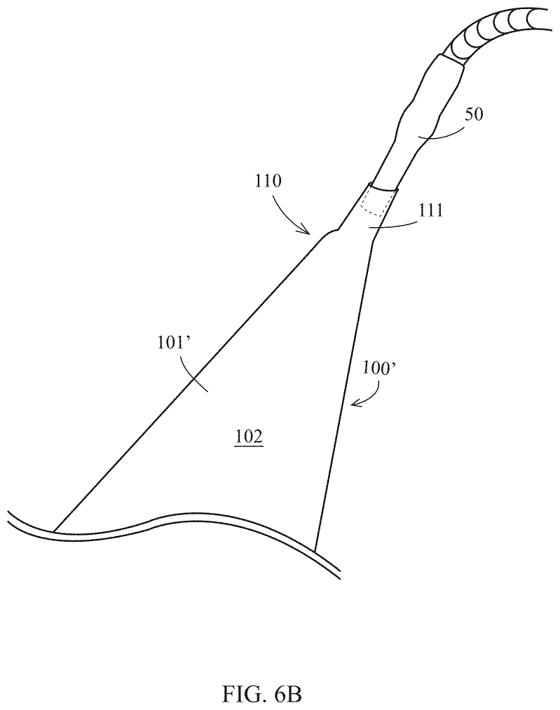

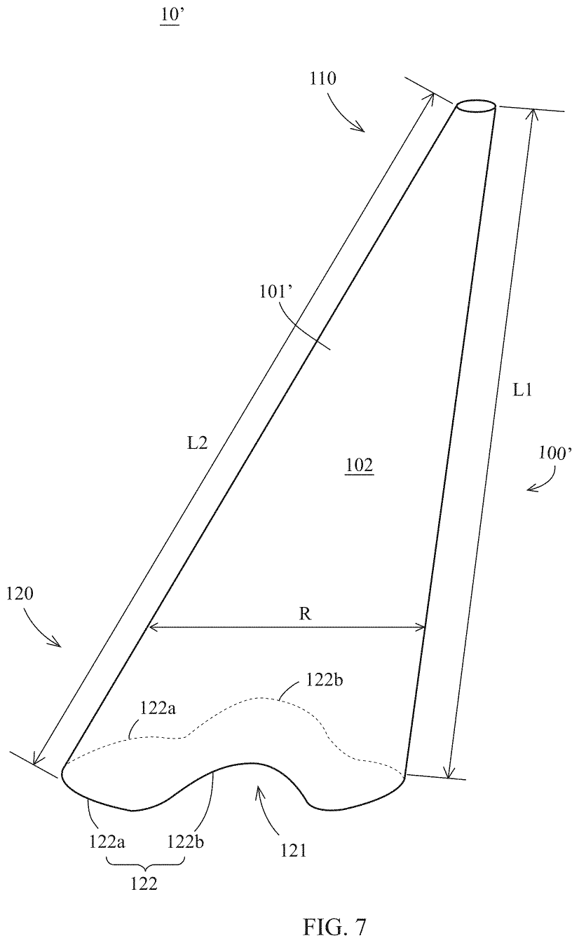

Continue to refer to FIG. 6A to FIG. 6B, the suction connecting portion 110 is located at an upper portion of the mask body 100′, and the operation portion 120 is located at a lower portion of the mask body 100′ relative to the suction connecting portion 110. The suction connecting portion 110 may further comprise a tube 111, and the tube 111 has a diameter r decreasing upward along an axial direction of the tube 111 so that the tube 111 assumes a shape of a cone so as to be universally connected with a long pipe 50 of the existing suction device (not shown) used in dental medical treatment. That is, one end of the long pipe 50 may be sleeved on the tube 111 (or the tube 111 may be sleeved on the long pipe 50), and then pressure is applied against each other with a common hand force so that the long pipe 50 and the tube 111 may be easily and tightly fitted with each other due to the change of the diameter r (meanwhile, an outward pulling force may be applied to each other for removal). Alternatively, as shown in FIG. 7, the suction connecting portion 110 may not have a tube 111, and the mask body 100′ may be tapered as a whole with a diameter R decreasing gradually from the operation portion 120 to the suction connecting portion 110, and thus the suction connecting portion 110 may still be conveniently connected with the long pipe 50. With the connection between the suction connecting portion 110 and the long pipe 50, the internal space 102 will form a negative-pressure space due to suction, and thus droplets, aerosols and liquid splashes from the oral area will be directly sucked into the suction device after approaching the internal space 102, and will not escape into the environment. When the aforesaid suction connecting portion 110 (with or without the tube 111) is connected with the long pipe 50, an elastic hose may be optionally placed between the suction connecting portion 110 and the long pipe 50 to increase the tightness and avoid gas leakage. Alternatively, after the suction connecting portion 110 is connected with the long pipe 50, an elastic hose is sleeved outside the connecting portion to achieve the same effect (as shown in FIG. 4). The elastic hose may be a silicone, rubber hose or an O-shaped ring or the like.

The operation portion 120 of the mask body 100, 100′ (hereinafter described by the mask body 100′) may comprise at least one opening 121, the opening 121 is irregular, has an outer edge 122 which is streamlined (generally wavy when spread out) and a portion thereof extends toward the suction connecting portion 110. The outer edge 122 may be approximately inverted U-shaped when viewed from the side of the mask body 100′, and may be oval or circular when viewed from the bottom of the mask body 100. When the mask body 100′ is used to cover the patient's oral area, parts 122a of the outer edge 122 may touch the face (which may include areas such as philtrum, cheeks, chin, etc.) of the patient, and the touched parts 122a are spaced apart from each other or opposite to each other. Another untouched part 122b is arranged between the parts 122a so that the parts 122a are staggered with the another part 122b, and the another part 122b further extends toward the suction connecting portion 110. Thus, a distance exists between the outer edge 122 and the face, through which several fingers of the medical personnel or an instrument used may pass, and thus the oral area is accessible for further oral examination. The streamlined outer edge 122 may avoid scratching of the hands of medical personnel, but it is not limited thereto. The outer edge 122 may also completely touch the face of the patient so that the touched portion forms a closed ring. At this time, the operating portion 120 may have at least one opening disposed above the outer edge 122 and separated from the outer edge 122, and is preferably located at the position corresponding to the part 122b (not shown), and the same effect can still be achieved. It shall be noted that, touching the face of the patient mentioned above may mean indirect touching, such as providing a hole towel or a rubber dam between the outer edge 122 and the patient (e.g., the trapezoid as shown in FIG. 1 and FIG. 4), or keeping a closer distance from the patient without touching the patient in order to avoid discomfort of the patient, while the another part 122b keeps a farther distance from the patient.

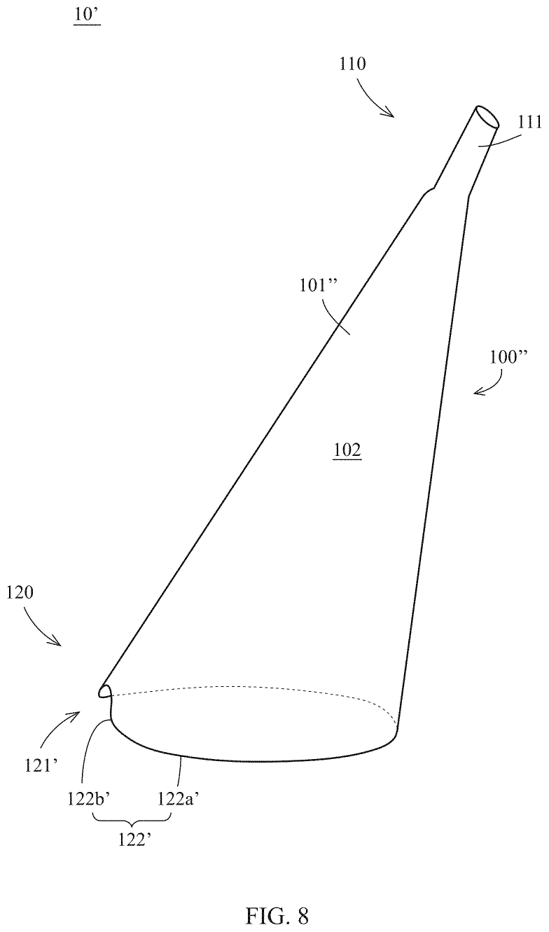

As shown in FIG. 8 and FIG. 9, which are respectively a schematic perspective view and a schematic spread-out view of the opening of the assist device 10′ located at different positions according to the second preferred embodiment of the present invention. The mask body 100″ comprised in the assist device 10′ may have the same technical features as the above mask body, and the technical contents applicable to each other are not repeated herein. The operation portion 120 of the mask body 100″ comprises an opening 121′ with an outer edge 122′, and the outer edge 122′ is streamlined and a portion thereof extends to the suction connecting portion 110. When the mask body 100″ is used to cover the oral area of the patient, the opening 121′ corresponds to the philtrum of the patient, the part 122b′ of the outer edge 122′ may abut against the patient, and the part 122a′ naturally creates a distance and space with the patient's face (which may include areas such as cheeks, chin, etc.), through which the doctor touches the patient's mouth. As compared to the first preferred embodiment, the part 122b′ of the outer edge 122′ of this embodiment is closer to the patient, i.e., extends a shorter distance to the suction connecting portion 110, so as to better achieve the above effect.

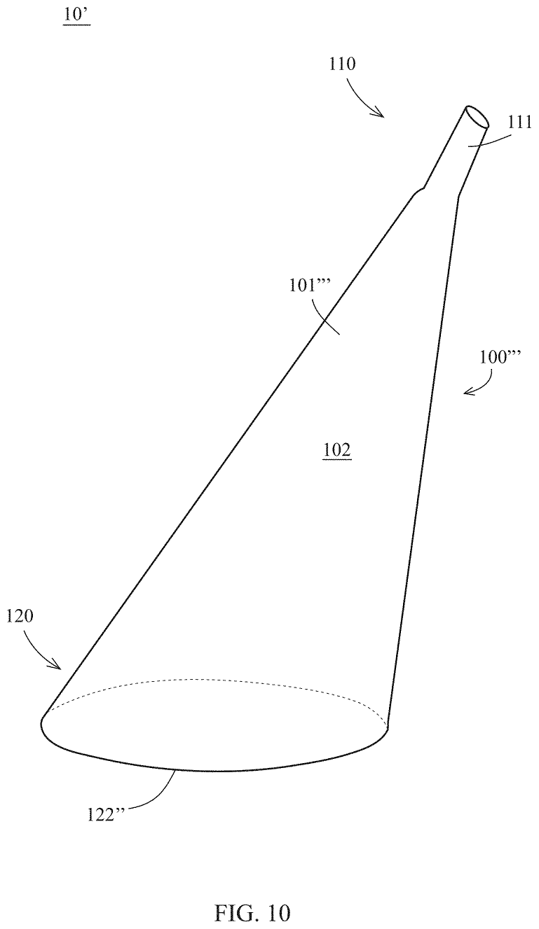

Please refer to FIG. 10, which is a schematic perspective view of the assist device 10′ without an opening according to the second preferred embodiment of the present invention. The mask body 100′″ comprised in the assist device 10′ may have the same technical features as the above mask body, and the technical contents applicable to each other are not repeated herein. The operation portion 120 of the mask body 100′″ does not comprise an opening, and the operation portion 120 may have an elliptical or circular outer edge 122″. When using the mask body 100″, one portion of the outer edge 122″ touches the face of the patient (which may be areas such as the philtrum, cheeks or chin, etc.), and other portions of the outer edge 122″ naturally create a distance with the patient, so that the doctor may touch the patient's mouth through the space between the outer edge 122″ and the face of the patient.

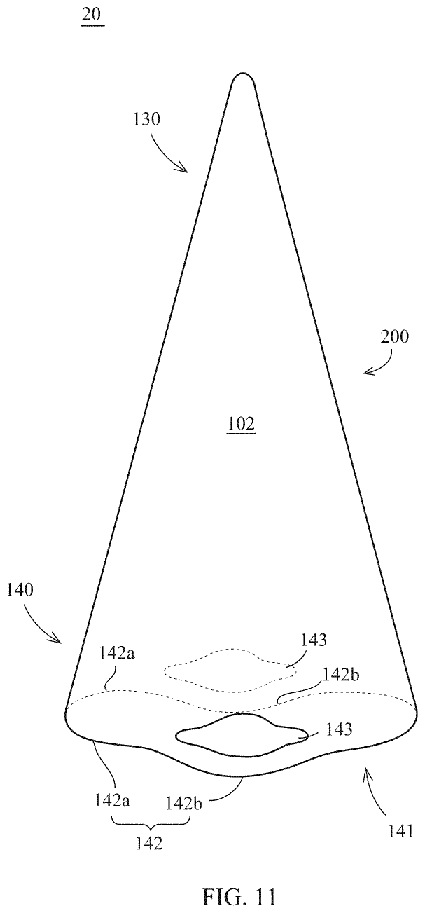

An assist device 20 according to a third preferred embodiment of the present invention is as shown in FIG. 11, and the mask body 200 of the assist device 20 may have the same technical features as the above mask body, and the technical contents applicable to each other are not repeated herein. The mask body 200 may be applied to throat examination and treatment in dentistry and otolaryngology or the like, and comprises a closed end 130 and an open end 140. The closed end 130 may have the same shape as the suction connecting portion 110 so that it is convenient for the patient to hold, fix the mask body 200 and observe the oral cavity, but it is not limited thereto. The open end 140 may comprise an opening 141, and the opening 141 may be optionally elliptical or circular and has the size corresponding to the oral area of the patient. The opening 141 has an outer edge 142 which may be streamlined as in the first and second embodiments. For example, the outer edge 142 may have parts 142a corresponding to the philtrum and chin of the patient, and parts 142b extending downward and corresponding to the cheeks and mouth corners on both sides of the patient, so as to better conform to the facial curve when the patient opens his/her mouth. In this way, when the mask body 200 is used to cover the oral area, the outer edge 142 may approach or closely abut against the face of the patient to prevent droplets and splashes of the oral area from leaving the mask body, but it is not limited thereto. The open end 140 may also comprise a plurality of openings (openings 141 and openings 143) so that medical personnel can still touch the oral cavity (e.g., using a tongue depressor) through the openings 143 for examination while the oral area is masked.

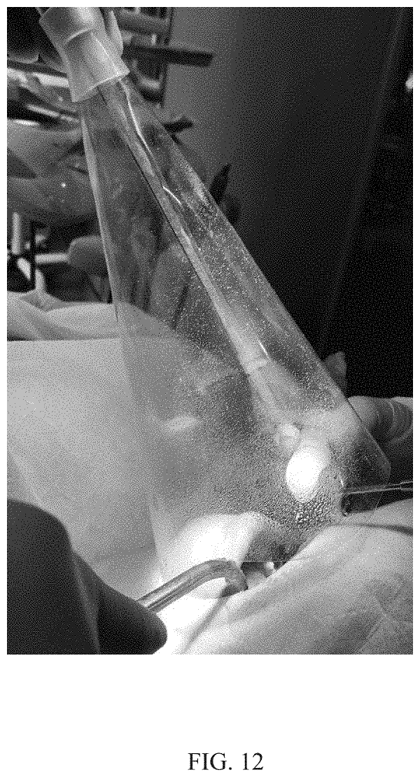

According to the above descriptions, comparing with the dental medical process in the past, droplets, aerosol, debris and splashing liquid may spread to the hands, chest and face of the doctor and assistant, and upper body of the patient, that exposing the medical personnel and patients to a lot of risks. The oral medical assistive device of the present invention comprises a mask body with is at least partially transparent so that medical personnel may cover the oral area of the patient at low cost when treating and examining the oral, thereby significantly limiting the splash range to the mask body (as shown in FIG. 12) and preventing droplets, aerosols, splashes or the like from spreading freely in the environment to protect both medical personnel and patients. The oral medical assistive device may further comprise a suction connecting portion and an operation portion, wherein the suction connecting portion may be further connected with a medical suction device, and a negative-pressure space is formed in the mask body by the suction of the suction device, thereby effectively absorbing droplets and aerosols. The operation portion may comprise at least one opening for the medical personnel to touch the oral cavity of the patient with hands thereof or instruments for examination. A mask body comprised in the oral medical assist device of the present invention may also comprise a closed end to prevent droplets and splashes of the oral area from leaving the mask body.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. An oral medical assist device adapted for covering an oral area of a patient, comprising:
a mask body, being at least partially transparent and defining an internal space, comprising a suction connecting portion and an operation portion, the suction connecting portion and the operation portion being configured to be opposite to each other, the operation portion comprising at least one opening;
wherein the suction connecting portion is configured to connect to a medical suction device to absorb objects and gases in the internal space while the mask body covers the oral area of the patient.

2. The oral medical assist device of claim 1, wherein the whole mask body is made of a transparent material and has a thickness that is not greater than 3.0 mm and not less than 0.1 mm.

3. The oral medical assist device of claim 1, wherein the suction connecting portion comprises a tube, and a diameter of the tube decreases gradually along an axial direction.

4. The oral medical assist device of claim 1, wherein the at least one opening has an outer edge, the outer edge is streamlined, and a portion of the outer edge extends toward the suction connecting portion.

5. The oral medical assist device of claim 1, wherein the mask body has a shape of an oblique cone.

6. An oral medical assist device adapted for covering an oral area of a patient, comprising:
a mask body, being at least partially transparent and defining an internal space, comprising a closed end and an open end opposite to the closed end;
wherein an outer edge of the open end of the mask body abuts against a face of the patient when the mask body covers the oral area, so that droplets from the oral area will not be spread outside of the mask body.

7. The oral medical assist device of claim 6, wherein the mask body is made of a transparent material and has a thickness that is not less than 0.1 mm.

8. The oral medical assist device of claim 6, wherein the mask body has a shape of an oblique cone.

9. The oral medical assist device of claim 6, wherein the open end may comprise at least two openings.
